(19) United States
(12) Patent Application Publication
(10) Pub. No.: US 2025/0221759 A1
(43) Pub. Date: Jul. 10, 2025

(54) MULTI-FACTOR ENFORCEMENT OF SINGLE-USE FOR DISPOSABLE RF DEVICES

(71) Applicant: Boston Scientific Scimed, Inc., Maple Grove, MN (US)

(72) Inventors: Steven Kinio, Pickering (CA); Deep Patel, Waterdown (CA); Emily Au, Mississauga (CA); MIchael Same, Mississauga (CA)

(21) Appl. No.: 19/008,009

(22) Filed: Jan. 2, 2025

Related U.S. Application Data
(60) Provisional application No. 63/617,532, filed on Jan. 4, 2024.

(51) Int. Cl. A61B 18/14; A61B 18/00; A61B 18/12

(52) U.S. Cl. CPC A61B 18/1477; A61B 18/1206; A61B 2018/00988

(57) ABSTRACT

An electrosurgical puncture device configured for use with an electrosurgical generator in an electrosurgical puncture procedure. The device includes a connector configured to couple the device to the electrosurgical generator, the connector including a radiofrequency (RF) conductor to receive an RF puncture signal provided by the electrosurgical generator. The device includes a tangible storage medium electrically coupled to the connector and includes data for use with the electrosurgical generator in the electrosurgical puncture procedure. The tangible storage medium also includes an asymmetric signature mechanism including a digital signature generated from a hash value of the data signed with a private key, a usage stamp having an associated generator code field and a timestamp field in a write protectable region of the storage medium, and a challenge response mechanism including a challenge engine. The electrosurgical generator includes a public key and provides the challenge.

### CROSS REFERENCE TO RELATED APPLICATIONS

This application claims priority to U.S. Provisional Patent Application No. 63/617,532 entitled “MULTI-FACTOR ENFORCEMENT OF SINGLE-USE FOR DISPOSABLE RF DEVICES,” filed Jan. 4, 2024, which is hereby incorporated by reference in its entirety.

### TECHNICAL FIELD

The present disclosure relates to medical devices and systems for use in percutaneous or interventional procedures including surgery such as electrophysiology procedures. More specifically, this disclosure relates to electrosurgical devices, such as single-use transseptal puncture devices that couple to and provide information to electrosurgical units, such as radiofrequency (RF) generators, from a non-transitory memory device.

### BACKGROUND

Catheters are often used to provide general access into a patient's body using minimally invasive techniques. In some examples, a catheter can be used to create a channel through a region of the body. For instance, punctures in tissues can provide access for medical tools used in various medical interventions. In one example, a pericardium layer of a patient can be punctured to provide for epicardial access, such as to create an access point to insert tools for epicardial ablation. In another example, electrosurgical devices are applied to remove accumulation of atheromatous material on the inner walls of vascular lumens, which results in atherosclerosis. In one technique, an electrosurgical device is applied to puncture through the vascular occlusion without affecting the vessel walls. Another example is a transseptal puncture in a cardiac procedure. The left atrium is a difficult cardiac chamber to reach percutaneously. Although the left atrium can be reached via the left ventricle and mitral valve, the catheter is manipulated through two U-turns, which can be cumbersome. the transseptal puncture is a technique of creating a small surgical passage through the atrial septum, or wall in the heart between the left and right atrium, through which a catheter can be fed. The atrial septum is punctured and dilated via tools. The transseptal puncture permits a direct route to the left atrium via the atrial septum and systematic venous system. Increasing larger and complex medical devices can be passed into the left atrium.

Punctures, such as transseptal punctures, can be performed with the aid of guidewires having electrodes energized with a suitable power source such as an electrically coupled power generator to provide the source of RF energy in a manner like other electrosurgical devices. Typical electrosurgical devices apply an electrical potential difference or a voltage difference between an active electrode and a return electrode on a patient's grounded body in a monopolar arrangement or between an active electrode and a return electrode on the device in bipolar arrangement to deliver the RF energy to the area where tissue is to be affected. Electrosurgical devices pass RF energy through tissue between the electrodes to puncture tissue with plasma formed on the energized electrode. Tissue that contacts the plasma experiences a rapid vaporization of cellular fluid to produce a cutting effect. Electrical energy can be applied to the electrodes either as a train of high frequency pulses or as a continuous signal typically in the radiofrequency (RF) range to perform the cutting or puncturing techniques.

### SUMMARY

In Example 1, an electrosurgical puncture device, the electrosurgical puncture device configured for use with an electrosurgical generator in an electrosurgical puncture procedure, the electrosurgical puncture device comprising: a connector configured to mechanically and electrically couple the electrosurgical puncture device to the electrosurgical generator, the connector including a radiofrequency (RF) conductor to receive an RF puncture signal provided by the electrosurgical generator; a tangible storage medium electrically coupled to the connector, the storage medium comprising: data configured for use with the electrosurgical generator in the electrosurgical puncture procedure; an asymmetric signature mechanism including a digital signature generated from a hash value of the data signed with a private key, the electrosurgical generator including a public key of the asymmetric signature mechanism to verify the hash value; a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; and a challenge-response mechanism including a secret in an unreadable region of the storage medium and a challenge engine, the challenge engine configured to calculate a response with the secret and a challenge received from the electrosurgical generator.

In Example 2, the electrosurgical puncture device of Example 1, comprising a transseptal puncture device.

In Example 3, the electrosurgical puncture device of Example 2, wherein the transseptal puncture device includes one of a transseptal puncture guidewire and a cable couplable to the transseptal puncture guidewire.

In Example 4, the electrosurgical puncture device of any of Examples 1-3, wherein the tangible storage medium includes a secure erasable electronic programmable read only memory (EEPROM).

In Example 5, the electrosurgical puncture device of any of Examples 1-4, wherein the data includes default settings for the electrosurgical generator.

In Example 6, the electrosurgical puncture device of any of Examples 1-5, wherein the digital signature and secret are generated at manufacture of the electrosurgical puncture device.

In Example 7, the electrosurgical puncture device of any of Examples 1-6, wherein the tangible storage medium includes a plurality of at least three enforcement mechanisms including the asymmetric signature mechanism, the usage stamp mechanism, and the challenge-response mechanism.

In Example 8, the electrosurgical device of any of Examples 1-7, wherein the tangible storage medium includes three enforcement mechanisms.

In Example 9, the electrosurgical puncture device of any of Examples 1-7, further comprising a usage count mechanism having a usage amount value in the write protectable region of the storage medium.

In Example 10, the electrosurgical puncture device of Example 9, wherein the usage amount value is configured to be one of incremented and decremented with each electrosurgical puncture procedure.

In Example 11, the electrosurgical puncture device of any of Examples 1-10, wherein the electrosurgical generator is configured to apply the asymmetric signature mechanism, the usage stamp mechanism, and the challenge-response mechanism, and permit the electrosurgical puncture procedure to proceed if the electrosurgical puncture device is valid.

In Example 12, the electrosurgical puncture device of Example 11, wherein the electrosurgical generator applies two or more of the asymmetric signature mechanism, the usage stamp mechanism, and the challenge-response mechanism concurrently.

In Example 13, the electrosurgical puncture device of any of Examples 11 and 12, wherein the electrosurgical generator is configured to apply additional enforcement mechanisms.

In Example 14, the electrosurgical puncture device of any of Examples 1-13, wherein the electrosurgical puncture device is included in an electrosurgical puncture assembly including a delivery component associated with the electrosurgical puncture device.

In Example 15, the electrosurgical puncture device of any of Examples 1-14, wherein the electrosurgical puncture device is included in an electrosurgical puncture system including the electrosurgical generator.

In Example 16, an electrosurgical puncture device, the electrosurgical puncture device configured for use with an electrosurgical generator in an electrosurgical puncture procedure, the electrosurgical puncture device comprising: a connector configured to mechanically and electrically couple the electrosurgical puncture device to the electrosurgical generator, the connector including a radiofrequency (RF) conductor to receive an RF puncture signal provided by the electrosurgical generator; a tangible storage medium electrically coupled to the connector, the storage medium comprising: data configured for use with the electrosurgical generator in the electrosurgical puncture procedure; an asymmetric signature mechanism including a digital signature generated from a hash value of the data signed with a private key, the electrosurgical generator including a public key of the asymmetric signature mechanism to verify the hash value; a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; and a challenge-response mechanism including a secret in an unreadable region of the storage medium and a challenge engine, the challenge engine configured to calculate a response with the secret and a challenge received from the electrosurgical generator.

In Example 17, the electrosurgical puncture device of Example 16, comprising a transseptal puncture device.

In Example 18, the electrosurgical puncture device of Example 17, wherein the transseptal puncture device includes one of a transseptal puncture guidewire and a cable couplable to the transseptal puncture guidewire.

In Example 19, the electrosurgical puncture device of Example 16, wherein the tangible storage medium includes a secure erasable electronic programmable read only memory (EEPROM).

In Example 20, the electrosurgical puncture device of Example 16, wherein the data includes default settings for the electrosurgical generator.

In Example 21, the electrosurgical puncture device of Example 16, wherein the digital signature and secret are generated at manufacture of the electrosurgical puncture device.

In Example 22, the electrosurgical puncture device of Example 16, wherein the tangible storage medium includes a plurality of at least three enforcement mechanisms including the asymmetric signature mechanism, the usage stamp mechanism, and the challenge-response mechanism.

In Example 23, the electrosurgical device of Example 16, wherein the tangible storage medium includes three enforcement mechanisms.

In Example 24, the electrosurgical puncture device of Example 16, further comprising a usage count mechanism having a usage amount value in the write protectable region of the storage medium.

In Example 25, the electrosurgical puncture device of Example 24, wherein the usage amount value is configured to be one of incremented and decremented with each electrosurgical puncture procedure.

In Example 26, the electrosurgical puncture device of Example 16, wherein the electrosurgical generator is configured to apply the asymmetric signature mechanism, the usage stamp mechanism, and the challenge-response mechanism, and permit the electrosurgical puncture procedure to proceed if the electrosurgical puncture device is valid.

In Example 27, the electrosurgical puncture device of Example 26, wherein the electrosurgical generator applies two or more of the asymmetric signature mechanism, the usage stamp mechanism, and the challenge-response mechanism concurrently.

In Example 28, the electrosurgical puncture device of Example 26, wherein the electrosurgical generator is configured to apply additional enforcement mechanisms.

In Example 29, the electrosurgical puncture device of Example 16, wherein the electrosurgical puncture device is included in an electrosurgical puncture assembly including a delivery component associated with the electrosurgical puncture device.

In Example 30, electrosurgical system configured for use in an electrosurgical puncture procedure, the electrosurgical system comprising: an electrosurgical generator configured to provide a radiofrequency (RF) puncture signal; and an electrosurgical puncture device, the electrosurgical puncture device configured for use with the electrosurgical generator in the electrosurgical puncture procedure, the electrosurgical puncture device comprising: a connector configured to mechanically and electrically couple the electrosurgical puncture device to the electrosurgical generator, the connector including an RF conductor to receive the RF puncture signal; a tangible storage medium electrically coupled to the connector, the storage medium comprising: data configured for use with the electrosurgical generator in the electrosurgical puncture procedure; an asymmetric signature mechanism including a digital signature generated from a hash value of the data signed with a private key, the electrosurgical generator including a public key of the asymmetric signature mechanism to verify the hash value; a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; and a challenge-response mechanism including a secret in an unreadable region of the storage medium and a challenge engine, the challenge engine configured to calculate a response with the secret and a challenge received from the electrosurgical generator.

In Example 31, the electrosurgical system of Example 30, wherein the electrosurgical puncture device is a transseptal puncture device including one of a transseptal puncture guidewire and a cable couplable to the transseptal puncture guidewire.

In Example 32, the electrosurgical puncture system of Example 31, wherein the transseptal puncture device is included in a transseptal puncture assembly including a delivery component associated with the transseptal puncture device.

In Example 33, an electrosurgical puncture device, the electrosurgical puncture device configured for use with an electrosurgical generator in an electrosurgical puncture procedure, the electrosurgical puncture device comprising: a connector configured to mechanically and electrically couple the electrosurgical puncture device to the electrosurgical generator, the connector including a radiofrequency (RF) conductor to receive an RF puncture signal provided by the electrosurgical generator; a tangible storage medium electrically coupled to the connector, the storage medium comprising: data configured for use with the electrosurgical generator in the electrosurgical puncture procedure; an asymmetric signature mechanism including a digital signature generated from a hash value of the data signed with a private key, the electrosurgical generator including a public key of the asymmetric signature mechanism to verify the hash value; a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; a challenge-response mechanism including a secret in an unreadable region of the storage medium and a challenge engine, the challenge engine configured to calculate a response with the secret and a challenge received from the electrosurgical generator; and a usage count mechanism having a usage amount value in the write protectable region of the storage medium.

In Example 34, the electrosurgical puncture device of Example 33, wherein the electrosurgical generator is configured to apply the asymmetric signature mechanism, the usage stamp mechanism, the challenge-response mechanism, and the usage count mechanism and permit the electrosurgical puncture procedure to proceed if the electrosurgical puncture device is valid.

In Example 35, the electrosurgical puncture device of Example 33, wherein the electrosurgical puncture device is included in an electrosurgical puncture system including the electrosurgical generator.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary electrosurgical system for performing an electrosurgical puncture, such as a transseptal puncture.

FIG. 2 is a schematic diagram of an example portion of an electrosurgical generator and an electrosurgical puncture device of the electrosurgical system of FIG. 1.

FIG. 3 is a schematic diagram illustrating an example memory map of a storage device of the electrosurgical puncture device of FIG. 2.

FIG. 4 is a block diagram illustrating an example process for use with the electrosurgical generator and electrosurgical puncture device of FIG. 2.

FIG. 5 is a map of features of the example process of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

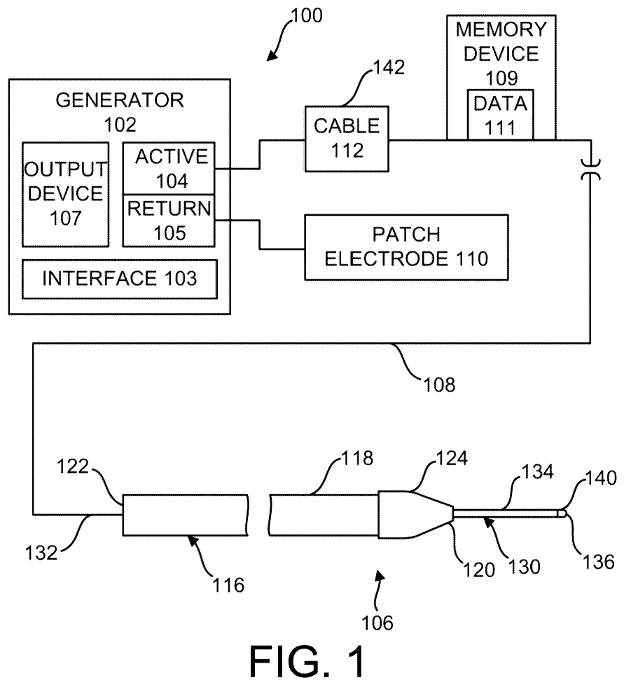

FIG. 1 illustrates an embodiment of an electrosurgical system 100 to facilitate vascular access to a heart and provide catheter positioning within cardiac anatomy. The embodiment of the medical system 100 includes an electrosurgical unit, such as an electrosurgical generator 102 and an electrosurgical device, such as an electrosurgical puncture device 106. In one example, the electrosurgical puncture device 106 includes an electrosurgical transseptal guidewire 108. In the illustration, the electrosurgical transseptal guidewire 108 is electrically coupled to the electrosurgical generator 102 via cable 112, such as to an active connector 104 on the electrosurgical generator 102. The electrosurgical generator 102 is configured to provide a source of energy, such as radiofrequency (RF) energy to the electrosurgical transseptal guidewire 108 via the cable 112. In some embodiments, the system 100 includes a ground pad electrode, or indifferent (dispersive) patch electrode 110 electrically coupled to the electrosurgical generator 102, such as to a return connector 105 on the electrosurgical generator 102, for use with the electrosurgical transseptal guidewire 108 in a monopolar configuration. In some embodiments, the electrosurgical transseptal guidewire is implemented in a bipolar configuration using a pair of electrodes on the guidewire and without a separate patch electrode.

The electrosurgical generator 102 is configured to provide the source of RF energy to the electrosurgical transseptal guidewire 108 for a puncture operation with the electrosurgical puncture device 106. The electrosurgical generator 102 includes an interface 103 including a set of user accessible controls, device connectors such as active connector 104 and a return connector 105, and an output device 107 such as a display device, speakers, and lights. In one embodiment, the device connectors 104, 105, can each include a receptable that includes a mechanical coupling to the electrosurgical puncture device 106 as well electrical contacts for electrically coupling the generator 102 to the electrosurgical puncture device 106. During a monopolar puncture operation of electrosurgical generator 102, a first electrode, often referred to as the active electrode, is provided with the electrosurgical puncture device 106 in general and with the transseptal guidewire 108 in the illustration while a second electrode, such as patch electrode 110, is typically located on the back, buttocks, upper leg, or other suitable anatomical location of the patient during surgery. In such a configuration, the patch electrode 110 is often referred to as a patient return electrode. An electrical circuit of RF energy is formed between the active electrode and the patch electrode 110 through the patient, which is used to puncture tissue at the active electrode. For example, RF energy for a puncture function in a monopolar mode may be provided at a relatively low voltage and a continuous current (100% on, or 100% duty cycle). At a power setting of 50 Watts for puncturing (although instantaneous power may be higher), voltage can range from approximately 164 to 400 volts root mean square (RMS). The electrosurgical generator 102 can include a plurality of functions and provide a programmed and custom settings via an interface and be couplable to a suite of electrosurgical devices in addition to the transeptal guidewire 108. In one example, the electrosurgical generator 102 provides RF energy to the active electrode as an alternating current having a frequency in the range of 100 KHz to 10 MHz. Typically, this energy is applied in the form of a continuous sinusoidal puncture signal. In some embodiments, the energy is applied in bursts of pulses. The individual pulses in each burst of a pulsed puncture signal typically each have a duration of 300 milliseconds with an interval between pulses of 700 milliseconds but can vary such as based on parameters of the connected electrosurgical puncture device 106. The actual pulses are often sinusoidal or square waves and bi-phasic, that is alternating positive and negative amplitudes.

In one example, the electrosurgical generator 102 provides the power to the electrosurgical puncture device 106, but the actual power level delivered to the electrosurgical puncture device 106 can be selected via controls on the electrosurgical puncture device 106 rather than controls on the electrosurgical generator 102. In another example, the electrosurgical generator 102 can be programmed to provide power levels within a selected range of power, and the electrosurgical puncture device 106 is used to select an output power level within the preprogrammed range. For instance, the electrosurgical generator 102 can be programmed to provide monopolar energy for a puncture function in a first range of power settings as well as voltage-based controls to target a specific voltage. The electrosurgical generator 102 can be programmed to provide monopolar energy for another function in a second range of power or voltage settings, which second range may be the same as, different than, or overlap the first range. In some embodiments, the user may then select the function and adjust the power or voltage setting within the range using controls on the electrosurgical puncture device 106 rather than using controls on the electrosurgical generator 102.

In one embodiment, the electrosurgical generator 102 can program and select particular controls, or ranges of controls, based on the particular configuration of the electrosurgical transeptal guidewire 108. The transseptal guidewire of the embodiment includes a memory device 109 (non-transitory memory) storing a set of parameters 111 associated with the transseptal guidewire 108. For instance, the data stored on the memory device 109 can include operating parameters regarding the set up and operation of the electrosurgical puncture device 106 in combination with the electrosurgical generator 102. The electrosurgical generator 102 is configured to read the parameters to program the controls to be suited for the associated transseptal guidewire. The memory device 109 can store the parameters 111 in various memory segments having lookup tables or other data structures to provide data to be loaded into a memory device in the electrosurgical generator 102 and read by a controller of the electrosurgical generator to affect operation. Example parameters 111 can include model number of the transseptal guidewire 108, acceptable power levels applied to the transseptal device 108, whether the transseptal device is configured for single use or multiple uses, as well as other parameters. In some embodiments, the electrosurgical generator 102 can be programmed to write to memory segments on the memory device 109 as well as read the memory device 109.

The illustrated electrosurgical puncture device 106 includes the electrosurgical transseptal guidewire 108 and a delivery component 116. While embodiments of the disclosure are described with reference to punctures in tissue with a transseptal guidewire for illustration, the features of the disclosure can be used with other electrosurgical devices including other transseptal surgical devices such as needle-based platforms. The delivery component 116 includes an elongated shaft 118 having a shaft distal tip 120. The elongated shaft 118 defines a longitudinally extending axial lumen 122. The electrosurgical transseptal guidewire 108 is adapted to be disposed within the lumen 122 and coupled to the RF energy source. In some embodiments, the delivery component 116 can include an elongated sheath, and the electrosurgical transseptal guidewire 108 is disposed within the sheath. In another embodiment, the delivery component 116 can include a dilator/sheath assembly, and the electrosurgical transseptal guidewire 108 is disposed within the dilator/sheath assembly. For instance, the elongated shaft 118 includes a distal tapered portion 124 with an enlargement of cross-sectional area with respect to the shaft distal tip 120. As the distal tapered portion 124 is passed through an aperture from the shaft distal tip 120, the enlargement of cross-sectional area dilates the aperture. The dilator can be configured as a straight dilator, as illustrated, or a curved dilator. The elongated shaft 118 can be made from various materials including insulative materials such as high-density polyethylene (HDPE). The shaft 118 and distal tip can include various materials such as metal hypotubes as well.

The electrosurgical transseptal guidewire 108 includes a puncture wire shaft 130 with a puncture wire proximal portion 132 and a puncture wire distal portion 134 having a puncture wire distal tip 136. The puncture wire distal tip 136 includes a puncture electrode 140 adapted to deliver the RF energy. The puncture electrode 140 is configured as the active electrode. The puncture wire proximal portion 132 includes an end connector 142 configured to electrically couple to cable 112 and receive an RF signal from the electrosurgical generator 102. In one example, the electrosurgical transeptal guidewire 108 can be mechanically and electrically coupled to and uncoupled from the cable 112 depending on whether the electrosurgical transeptal guidewire 108 is used as an electrosurgical puncture device or as an exchange rail, for instance. In such examples, the cable 112 can remain mechanically coupled to the active connector 104 receptacle and electrically coupled with electrical contacts to the electrosurgical generator. The transseptal guidewire 108 is configured to conduct the RF signal from the proximal portion 132 along the puncture wire shaft 130 to the electrode 140. In some embodiments, the puncture wire shaft 130 is constructed from an electrically conductive material having an insulative outer coating. In some embodiments, the electrically conductive material is a flexible, shape memory material such as a nickel titanium alloy or nitinol. The exposed electrode 140 is configured to apply the RF energy, such as to puncture tissue. In some embodiments of the electrosurgical device 106, a cable 112 is not used, and the electrosurgical puncture device 106 is directly coupled to, both mechanically and electrically, the electrosurgical generator 102. In such embodiments, the electrosurgical generator 102 can remain coupled to the electrosurgical puncture device 106 throughout the entire surgical procedure.

In the illustrated example, the electrosurgical transseptal guidewire 108 is configured as a multifunction conductive guidewire. For instance, the transseptal guidewire 108 can be used, without exchanges, as a guidewire, a transseptal puncture device, and as an exchange rail for delivering therapy sheaths. Such embodiments provide efficiencies to medical procedures as the transseptal guidewire 108 performs multiple functions and reduces the amount of device exchanges in the medical procedure. The transseptal guidewire 108 includes a distal tip 136 extendable from the delivery component distal end 120 such that the delivery component 116 is retractable from the patient over the guidewire 108 with the guidewire distal tip 136 disposed within the heart. The transseptal guidewire 108 is sufficiently thin and flexible to access the various chambers of the heart. The electrode 140 on the puncture wire distal tip 136 is operable to deliver RF energy to puncture the atrial septum from the right atrium, and the distal portion 134 of the puncture wire shaft 130 can be advanced through the puncture. Once advanced through the puncture and sufficiently extended from within the delivery component 136, the distal portion 134 is biased to form a coil for anchoring the transseptal guidewire 108 beyond the puncture. The delivery component 116 is retractable from the patient over the transseptal guidewire 108 with the distal tip 136 still disposed within the heart. The transseptal guidewire 108 can also support the installation of therapy devices to a therapy location in the patient's heart, such as tubular members or other catheters and for advancing other devices within the heart.

In an anticipated use of the system 100, the electrosurgical puncture device 106 is coupled to the RF generator 102, and if the electrosurgical puncture device 106 is to be configured in a monopolar mode, the patch electrode 110 is coupled to the patient. The RF generator 102 can receive data stored on a memory device 109 on the electrosurgical puncture device 106 and, in some examples, the RF generator can write to memory device 109. The RF generator 102 can be set to a puncture mode, such as an energy output of approximately 50 watts, and configured for use. In some examples, femoral access is obtained via a conventional percutaneous needle, and the transseptal guidewire 108 is inserted into the vasculature and advanced to the superior vena cava. The shaft distal tip 120 of the delivery component 116 is advanced over the proximal portion 132 of the guidewire 108, and the distal tapered portion 124 of the delivery component shaft 118 is advanced over the guidewire 108 to the superior vena cava. Under visualization, the distal tapered portion 124 is moved from the superior vena cava to the right atrial septum and then to the fossa ovalis of the heart. Once the delivery component distal tip 120 is confirmed at the fossa ovalis, the electrode 140 of the transseptal guidewire 108 is advanced from the delivery component distal tip 120. In one example, the exposed puncture electrode 140 of the transseptal guidewire 108 is extended a few millimeters from the delivery component distal tip 120 to tent the heart tissue, and the transseptal guidewire 108 can be locked in position with respect to the delivery component 116. Forward pressure is applied to the electrosurgical puncture device 106 and the transseptal guidewire 108 is actuated to apply the RF energy to the electrode 140 and puncture the fossa ovalis. The RF energy punctures the fossa ovalis and creates an aperture in the fossa ovalis. The transseptal guidewire 108 is unlocked from the delivery component 116, and the transseptal guidewire 108 is extended through the aperture. In general, the transseptal guidewire 108 is extended longitudinally for several millimeters prior to the distal portion 134 curving to assume a J-tip or pigtail shape and deflecting away from the atrial septum. The transseptal guidewire 108 can be advanced into the left atrium of the heart and anchored. In the embodiment of the delivery component 116 configured as the dilator/sheath assembly, the distal tapered portion 150 of a dilator, the distal tapered portion 124 is advanced into the puncture site to expand the aperture. The delivery component 116 can be retracted from the patient over the transseptal guidewire 108, and transseptal guidewire 108 can provide support for the installation of tubular members or other catheters and for advancing other devices within the heart.

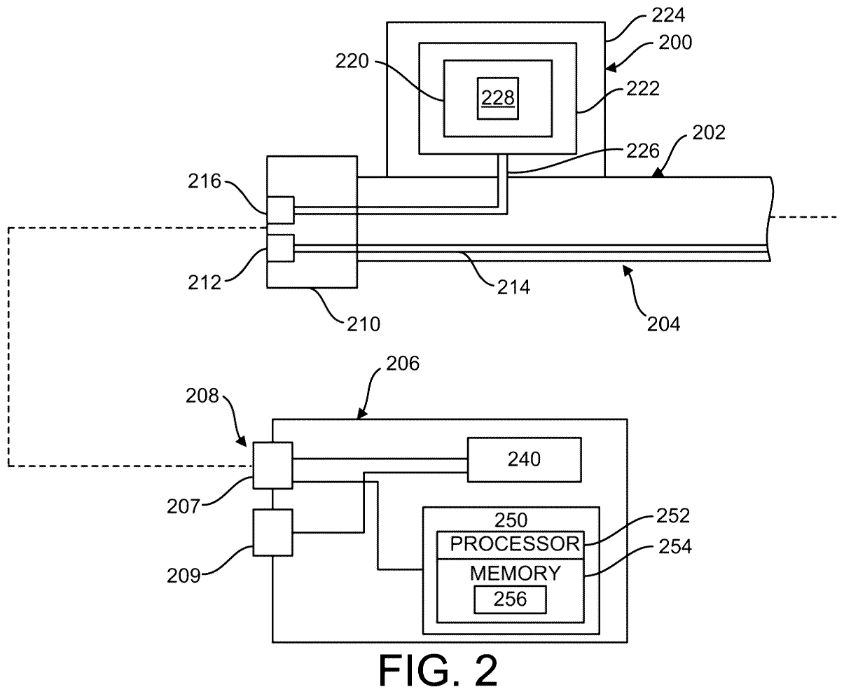

FIG. 2 illustrates an embodiment of a storage device 200 coupled to a proximal section 202 of an electrosurgical puncture device 204. The electrosurgical puncture device 204 can correspond with electrosurgical puncture device 106—such as with a cable 112, RF puncture assembly, RF transseptal guidewire, or combinations of electrosurgical puncture devices 204 such as the cable 112 combined with the transseptal guidewire 108 in some embodiments. FIG. 2 also illustrates an RF generator 206, which can correspond with the electrosurgical generator 102, couplable to the electrosurgical storage device 204 via receptacles 208 that can include active connector receptacle 207 and return connector receptacle 209.

The proximal section 202 includes a connector 210 configured to couple the electrosurgical puncture device 204 to the RF generator 206. In the illustrated embodiment, the connector 210 is configured to mechanically and electrically couple the electrosurgical puncture device 204 to the RF generator 206. The connector 210 includes an RF connector 212 coupled to an RF electrical lead 214 configured to couple to the electrosurgical generator 102 and receive a puncture signal such as from the RF generator 206. The RF electrical lead 214 is configured to electrically couple the RF connector 212 to the puncture electrode, such as puncture electrode 140. For instance, the RF connector 212 is configured to couple to the active connector 207 of the RF generator 206. In some embodiments, such as in an electrosurgical device 204 configured to operate in bipolar mode, the RF connector 212 includes a plurality of connectors and is configured to couple to the active connector receptacle 207 and the return connector receptacle 209 of the RF generator 206. In some embodiments, the RF electrical lead 214 is directly coupled to the puncture electrode. In other embodiments, the RF lead 214 is coupled to puncture electrode via another electrical connector, such as from cable 112 to electrosurgical transseptal guidewire 108. The connector 210 also includes an electrical data contact 216 configured to electrically couple the storage device 200 to the RF generator 206. For instance, the electrical data contact 216 can include a communication bus contact and a ground connection electrically coupled to the storage device 200. In some embodiments, the storage device 200 is integrally formed within the connector 210. In some embodiments, the storage device 200 is mechanically coupled to the connector 210 and disposed within the electrosurgical puncture device 204. In still other embodiments, the storage device 200 is mechanically coupled to the connector 210 via a tether.

In one illustrative embodiment, the storage device 200 includes a tangible storage medium 220 connected to a printed circuit board 222 forming a storage assembly. The storage medium 220 in the illustrated embodiment is enclosed in a housing 224 and electrically coupled, such as via conductive leads 226 attached to the printed circuit board 222, to the electrical contact 212. The tangible storage medium 220 in one embodiment includes an electronically erasable programmable read only memory (EEPROM) on which data 228 can be stored. In one embodiment, the data can include parameters 111. In one embodiment, the tangible storage medium 220 is a security EEPROM that includes a defense mechanism designed to protect against attacks with secure personalization features. In one embodiment, the secure EEPROM is configured in a serial architecture compatible with a four-wire Serial Peripheral Interface (SPI), a two-wire Inter-Integrated Circuit (I2C) or Universal Asynchronous Receiver/Transmitter (UART), or a single-wire 1-Wire protocol footprint. Other protocols now known or later developed are contemplated. In embodiments the secure EEPROM can include features such as user-programmable and irreversible protection modes including authentication, write and read protection, encryption, engines, and emulations. Other examples of non-transitory memory devices for use at the tangible storage medium 220 can include non-volatile memory device such as a read only memory (ROM), electronically programmable read only memory (EPROM), flash memory, non-volatile random access memory (NRAM) and a volatile memory device such as random access memory (RAM) or other memory device known or yet to be developed. Further, tangible storage medium 220 can include various combinations of one or both of non-volatile memory devices and volatile memory devices. In one embodiment, data 228 is provided serially via the communication bus to electrical contact 212.

The data 228 stored on tangible storage medium 220 can include one or more of a number of items for use with the RF generator 206 in an electrosurgical procedure. For example, data 228 can include an identifier, such as a model number or serial number and date of manufacture of the electrosurgical puncture device 204, and version number of the data 228. In some examples, the electrosurgical puncture device 204 is single-use device that is not intended to be used more than once. Data 228 can include features of the electrosurgical device 204 such as sensors operable on the electrosurgical puncture device 204. Other examples of data 228 can include default settings for the RF generator 206 for use with the electrosurgical puncture device 204 or limits on operating parameters, including voltages, currents, power, and temperature, of the electrosurgical puncture device 204. Data 228 can include calibrations and other information that can reduce firmware updates to the RF generators 206.

The RF generator 206 includes an RF output circuit 240 electrically coupled to the active and return connector receptacles 207, 209, and a controller 250 electrically coupled to at least the active connector receptacle 207. In one example, the RF output circuit 240 is configured to generate an RF puncture signal based on information provided from the data 228 and provide the RF puncture signal to the electrosurgical puncture device 204. The RF output circuit 240 can include a power supply to provide a direct current signal and can convert the direct current signal to an alternating current signal. The RF output circuit 240 can be configured to generate a plurality of voltages, waveforms having various duty cycles, peak voltages, crest factors, frequencies and other suitable signal features.

The controller 250 in embodiments includes a processor 252 operably connected to a memory device 254. The memory device 254 can store processor executable instructions configured to control the processor, such as a program 256. Examples of a memory device 254 can include a non-volatile memory device and a volatile memory device or other memory device. Memory device 224 can include various combinations of one or both of non-volatile memory devices and volatile memory devices. The processor 252 includes an output port that allows the processor 252 to control the output of or by the electrosurgical unit 206 according to a selected design. In some embodiments, the controller 250 includes a microprocessor or a logic processor or other control circuit such as a field programmable gate array. The controller 250 is coupled to the storage device 200 via an interface with the electrical data contact 216 of the electrosurgical puncture device 204 and is configured to read data 228 from the storage medium 220, process the data 228, and in some embodiments write to the storage medium 220.

Any combination of hardware and programming may be used to implement the functionalities of the electrosurgical unit 206. Such combinations of hardware and programming may be implemented in a number of different ways. For example, the programming for the electrosurgical unit 206 may be processor executable instructions stored on at least one non-transitory machine-readable storage medium, such as memory device 254 and the hardware may include at least one processing resource, such as processor 252, to execute those instructions. In some examples, the hardware may also include other electronic circuitry to at least partially implement at least one feature of electrosurgical unit 206. In some examples, the at least one machine-readable storage medium, such as a memory device 254, may store instructions that, when executed by the processor 252, at least partially implement some or all features of electrosurgical unit 206 and access data structures stored on a memory device coupled to the processor 252. In such examples, electrosurgical unit 206 may include the at least one machine-readable storage medium storing the instructions and the at least one processing resource to execute a method. The processor-executable instructions 256 may be in the form of an application, such as a computer application or module of a computer application. In other examples, the functionalities of electrosurgical unit 206 and method may be at least partially implemented in the form of electronic circuitry.

In many embodiments, data 228 is used to authenticate the electrosurgical puncture device to the electrosurgical generator and to enforce proper and intended application of the electrosurgical puncture device, which can enhance patient safety. Data 228 can be used to confirm that the electrosurgical puncture device 204 was developed, tested, and approved for use with the electrosurgical generator 102. Data 228 can be used to confirm that the electrosurgical puncture device was provided from an authorized manufacturer and conforms to the rigorous tests and specifications associated with the puncture device. Further, data 228 can be used to confirm that a single use electrosurgical puncture device is indeed new and never before used on a patient or in a surgical procedure. Unfortunately, data 228 stored on memory device 220 is subject to copying and modification by attackers, which can be used to create unauthorized puncture devices, puncture devices out of specification, puncture devices configured for unauthorized uses, and puncture devices configured to maliciously modify the software of electrosurgical generators and other equipment used in electrosurgical puncture systems.

Applicants have identified several vulnerabilities or vectors of attack of such data in electrosurgical puncture systems. Such vulnerabilities can be categorized as leading to (1) reuse of single use devices after a particular time or procedure with an RF generator, (2) reuse of single use device on multiple RF generators, (3) cloning of single use devices, (4) manufacture of counterfeit devices, (5) reverse engineering, software and firmware, and data, (6) tampering of data on the electrosurgical puncture device 204 or the RF generator. While many electrosurgical puncture systems include enforcement mechanisms to protect against vectors of attack, typically, such enforcement mechanisms either provide incomplete protections or are loaded onto capital devices that carry development costs and are subject to vulnerabilities if not updated.

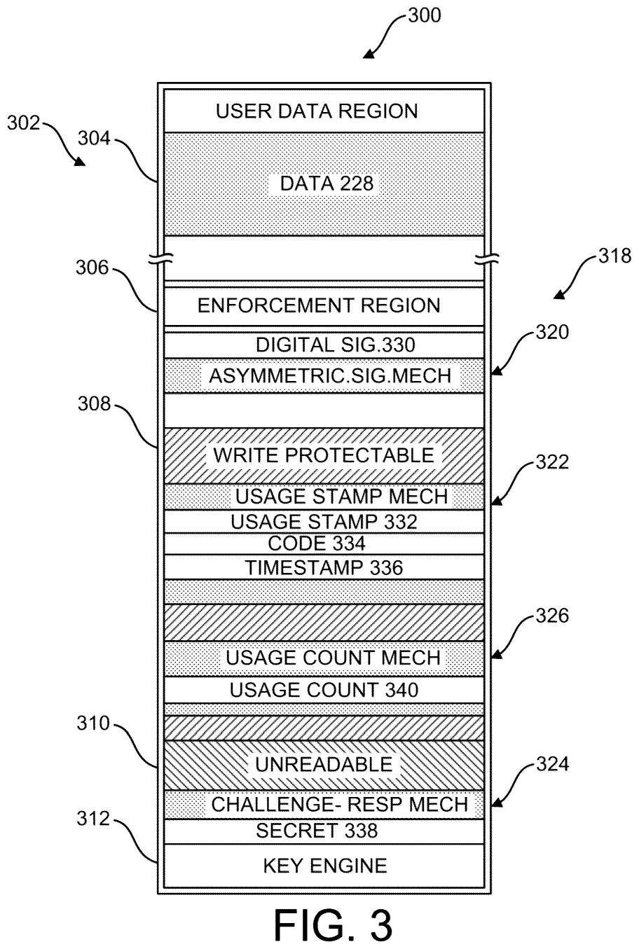

FIG. 3 illustrates an example memory map 300 of the tangible storage medium 220 in storage device 200. In the illustrated embodiment, the memory map 300 corresponds with a secure EEPROM tangible storage medium 220. Tangible storage medium 220 includes a plurality of memory regions 302 including a user data region 304 and an enforcement region 306. The user data region 304 that includes stored data or parameters used by the electrosurgical system, such as data 228 that can include identifiers, tables, parameters, and other information that can configure the electrosurgical generator 206 for use with the electrosurgical puncture device 204. The stored data 228 can be in a suitable format for use with the electrosurgical generator 206. The enforcement region 306 includes a write protectable region 308 and an unreadable region 310 (region including stored data cryptographically protected from discovery such as secure storage for secrets). Further, the enforcement region 306 is configured to include a symmetric key engine 312. For example, the symmetric key engine 312 can be configured to run a hash algorithm, such as Secure Hash Algorithm 3. Further, the secure EEPROM can include features to indicate whether the enforcement region has been tampered with, and whether the unreadable region 310 has been accessed or the write protectable region 308 has been written to.

The enforcement region 306 is programmed to include a plurality of at least three enforcement mechanisms 318 combined to provide a multi-factor authentication scheme for the electrosurgical puncture device 204. The enforcement mechanisms 318 include an asymmetric signature mechanism 320, a usage stamp mechanism 322 in the write protectable region 308, and a challenge-response mechanism 324 in the unreadable region 310 and key engine 312. In the illustrated example, the plurality of enforcement mechanisms 318 also includes an additional enforcement mechanism such as a usage count mechanism 326 in the write protectable region 308. In the illustrated embodiment, the asymmetric signature mechanism 320 is configured to store a digital signature 330, such as a digital signature created during manufacture of the storage device 200. The usage stamp mechanism 322 is configured to store and provide to the controller 250 a usage stamp 332 that can include an associated generator code 334 and a timestamp 336. In one example, the write protectable region 308 can be configured to permit a one-time write from the controller 250 to the usage stamp 322. The challenge-response mechanism 324 stores a secret 338 in the unreadable region 310 and employs the secret 338 with the key engine 312 to generate a response to the controller 250.

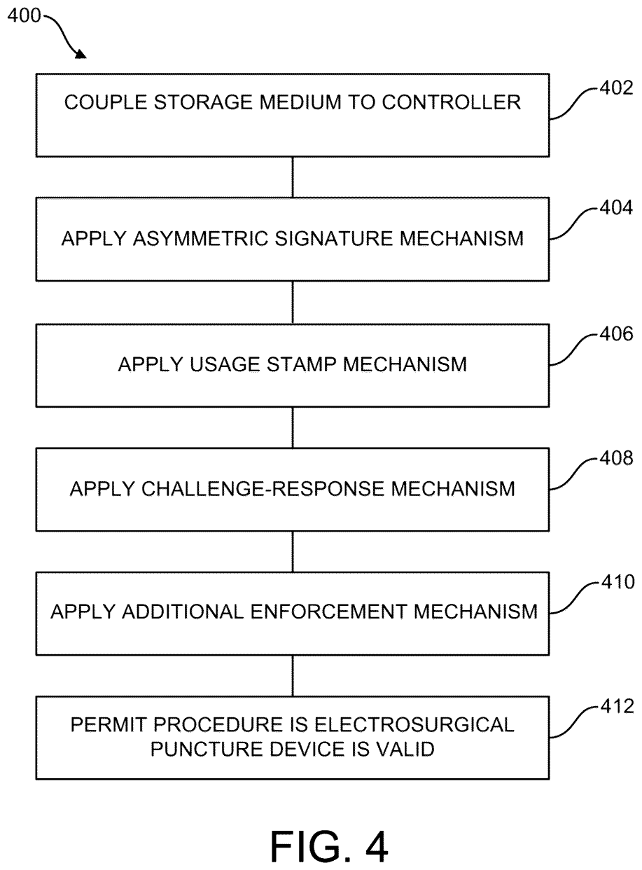

FIG. 4 illustrates an example method 400 for use with the RF generator 206 in combination with the electrosurgical puncture device 204 including the tangible storage medium 220. The electrosurgical puncture device 204 is attached to the RF generator 206, and the storage medium 220 is coupled to the controller 250 at 402. The controller 250 applies the asymmetric signature mechanism 320 at 404. the controller 250 employs the data 228 and digital signal 330 to determine if the data 228 is genuine and unaltered. The controller 250 applies the usage stamp mechanism at 406. The controller 250 accesses the usage stamp 332 that can include an associated generator code 334 and a timestamp 336 on the storage medium 220. The controller 250 applies the challenge-response mechanism at 408. The key engine 312 employs the secret 338 to generate a response to the controller 250. The controller 250 applies any additional enforcement mechanisms 318, such as a usage count mechanism 326 at 410. If the electrosurgical puncture device 204 and RF generator 206 combination passes the enforcement mechanisms 308, the electrosurgical puncture device 204 is determined valid, and the controller 250 permits an electrosurgical procedure with RF generator 206 in combination with the electrosurgical puncture device 204 to proceed at 412. Application of the enforcement mechanisms 308 can be applied concurrently or in any order.

The asymmetric signature mechanism 320 combines a hash value of the data 228 in data region 304 with asymmetric encryption to create a digital signature 330 at 404. For example, a hash algorithm is applied to genuine data 228 during manufacturing of the storage device 200 by an authorized manufacturer to create a hash value. Examples of hash algorithms create fixed sized strings of the data as hash values, or message digests. In general, hash algorithm repeatedly applied to a data set will yield the same message digest. The hash algorithm applied to a modified data set, however, will yield a different message digest.

The hash value created during manufacturing is encrypted with a private key also applied at manufacturing to create the digital signature 330. The private key is kept secret as part of manufacturing the storage device 200. The digital signature 330 is stored with the asymmetric signature mechanism 320 on the storage device 200. The asymmetric signature mechanism 320 is configured to provide the data 228 and the digital signature 330 together, such as in a file to the RF generator 206.

The RF generator 206 is configured to receive the data 228 and the digital signature 330 such as with the controller 250. For example, the controller 250 receives the data 228 and digital signature 330 when the electrosurgical puncture device 204 is set up for an electrosurgical puncture procedure. In one embodiment, the controller 250 receives data 228 and digital signature 330 when the electrosurgical puncture device 204 is connected to the RF generator 206 or when the RF generator 206 is started up with the connected puncture device 204. The controller 250 includes a public key stored in the memory 254 for use with the processor 252. The public key is associated with the private key. In one example, the public key can be stored on RF generators configured or authorized to operate with the electrosurgical puncture device 204.

In one embodiment, the controller 250 separates the received data 228 from the received digital signature 330. The controller 250 applies the public key to the digital signature 330 to decrypt the digital signature 330 and retrieve the original hash value of the data 228. If the public key can decrypt the digital signature 330, the controller 250 is able to verify that the data 228 is from the authorized manufacturer. The original hash value is stored in memory 254. The controller 250 can apply the hash algorithm used during manufacturing to the received data 228 to create a new hash value. The new hash value is compared to the original hash value. If the new hash value is the same as the original hash value, the controller is able to verify that the data 228 is genuine and unaltered.

The usage stamp mechanism 322 in the write protectable section 308 of the storage medium 220 is configured to provide an input to the RF generator 206 that ties the electrosurgical puncture device 204 to the RF generator 206 at 406. Additionally, the usage stamp mechanism 322 provides for time-based expiration of life of the electrosurgical puncture device 204. For example, the controller 250 is configured to receive a usage stamp 332 from the storage device 220 including an associated generator code 334, such as a serial number or other identifier associated with a particular RF generator 206, and a timestamp 336 from the write protectable region 308 when the electrosurgical puncture device 204 is set up for an electrosurgical puncture procedure. In one example, the associated generator code 334 can be a weak identifier, such as the serial number of the particular RF generator. In one embodiment, the controller 250 provides the associated generator code 334 and timestamp 336 from the write protectable region 308 when the electrosurgical puncture device 204 is connected to the RF generator 206 or when the RF generator 206 is started up with the connected puncture device 204. In one embodiment, the RF generator 206 can apply the write protection following writing of the code 334 and timestamp 336.

If the usage stamp 332 does not have an informational input to provide to the controller 250, or the usage stamp 332 provided is associated with blank fields, such as blank fields for the associated generator code 334 and timestamp 336, the controller 250 can determine that the corresponding electrosurgical puncture device 204 is new and never before used. The controller 250 permits operation of the RF generator 206 with the attached electrosurgical puncture device 204. The controller 250 is also configured to write appropriate data to the associated generator code 334 and timestamp 336 to the usage stamp mechanism 322. In one example, the controller 250 is configured to write the serial number to the associated generator code 334 and the current time to the timestamp 336 on the storage device 220. The write protectable region 308 of the storage medium 220 reduces the likelihood of the usage stamp 332 being overwritten or erased.

If the usage stamp mechanism 322 does have an information input to provide to the RF generator, such as a serial number and time, the controller 250 determines whether the associated generator code 334 matches the code of the RF generator 206 and whether the present time is within an acceptable threshold limit of the timestamp 336. For example, the associated generator code 334 is unique to the particular RF generator, and other RF generators, including same-model RF generators, have a different code than the associated generator code 334. Also, the acceptable threshold can be a period of time, such as eight hours. The period of time can be added to the timestamp 336 and compared to the current time, such as the time of startup. Use of the electrosurgical puncture device 204 can be configured to expire at a time after the period of time added to the timestamp. If the present time is within the acceptable limit of the timestamp 336 and the associated generator code 334 matches the code of the RF generator, the RF generator 206 is configured to operate with the attached electrosurgical puncture device 204. If, however, the present time is past the acceptable limit of the timestamp 336 or the associated generator code 334 does not match the code of the RF generator, the RF generator 206 is configured to not operate with the attached electrosurgical puncture device 204.

The usage stamp mechanism 322 ties the electrosurgical puncture device to a single therapy site at a given time. For example, the usage stamp mechanism 322 reduces the likelihood of reuse of the associated electrosurgical device 204. The information related to expiration and associated RF generator are kept on the storage device 200, which reduces development overhead for the RF generator 206, and avoids strategies such as erasing signatures used to attack electrosurgical systems.

The challenge-response mechanism 324 stores the secret 338 in the unreadable region 310 and employs the secret 338 with the key engine 312 to generate a response to the controller 250 at 408. Prior to use of the electrosurgical puncture device 204, the controller 250 provides a challenge, or challenge key, to the tangible storage medium 220, such as at startup or when the electrosurgical puncture device 204 is connected to the RF generator 206. The key engine 312 receives the challenge from the controller 250 and the secret 338 from within the storage device 220 and calculates a response based on the challenge and secret 338. The secret 338 is in the unreadable region 310, which is not accessible from outside of the tangible storage device 220 after the unreadable region 310 has been programmed with the secret 338 during manufacture. Accordingly, the unreadable region 310 is neither readable nor writeable from a device after the electrosurgical puncture device 204 has programmed and left the manufacturing factory. The key engine 312 provides the calculated response to the controller 250. The controller 250 determines whether the received calculated response is correct. If the received calculated response is correct, the RF generator 206 can proceed with the electrosurgical puncture device 204 in the procedure. If the received calculated response is not correct, the RF generator 206 does not allow use of the electrosurgical puncture device 204 in the procedure.

In one embodiment, the controller 250 computes an authorization response based on same challenge and secret. For instance, the controller 250 can be programmed to include the same secret at manufacture, which is stored in a protected area of memory 254. The controller 250 receives the calculated response from the electrosurgical puncture generator 204 and compares the calculated response to the authorization response. If the values match, the electrosurgical puncture device 204 is determined to be valid, and the RF generator 206 can proceed with the electrosurgical puncture device 204 in the procedure. If the received calculated response does not match the authorization response, the electrosurgical puncture device 204 is determined to be invalid, and the RF generator 206 does not allow use of the electrosurgical puncture device 204 in the procedure.

The challenge-response mechanism 324 is particularly suited to protecting the tangible storage device from cloning, as the secret 338, as well as the unreadable region 310 in general, is not available to be read by cloners of the electrosurgical puncture device 204.

In some embodiments, the enforcement region 306 is programmed to include more than the three enforcement mechanisms 318 of the asymmetric signature mechanism 320, the usage stamp mechanism 322, and the challenge-response mechanism 324 combine to provide a multi-factor authentication scheme for the electrosurgical puncture device 204. The enforcement mechanisms 318 can include other security features. For instance, the enforcement mechanisms 318 can include the usage count mechanism 326 having a usage amount value 340 in the write protectable region 308. In one embodiment, the controller 250 can adjust the usage amount value 340, such as increments or decrements the usage amount value 340. The controller 250 then reads the usage amount value 340, and if the usage amount value corresponds with a suitable usage value of a set of suitable usage values (or is within a threshold limit), the electrosurgical puncture device 204 is determined to be valid. For example, if the adjusted usage value 340 has been decremented to zero, or incremented to a selected amount, the electrosurgical puncture device 204 has been used more than an authorized amount. In one example, the usage amount value 340 is programmed at the factory to be zero and set of suitable usage values only includes the value one. Once the suitable usage value is incremented to be one, such as during the first use of the electrosurgical device 204, the usage amount value can no longer be incremented to yield a suitable usage value. The electrosurgical puncture device 204 is no longer valid and has expired.

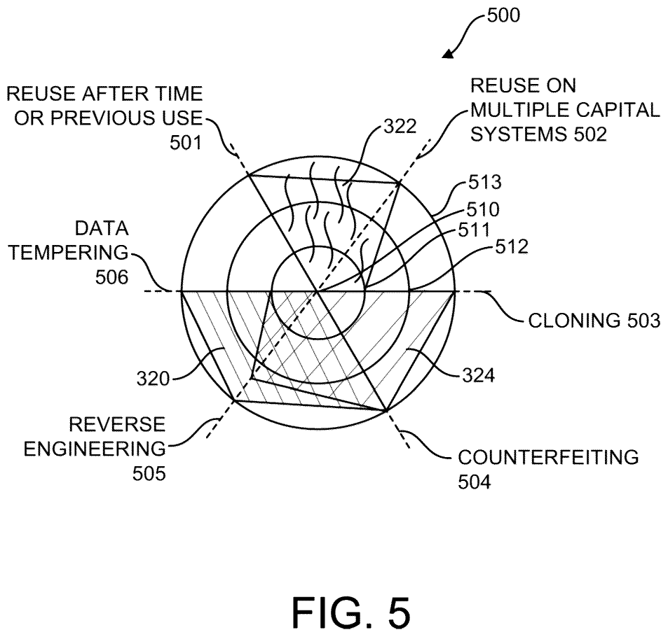

FIG. 5 illustrates a map 500 of the relative strengths of the asymmetric signature mechanism 320, the usage stamp mechanism 322, and the challenge-response mechanism 324 against the six determined vulnerabilities of the electrosurgical puncture devices, such as single-use puncture devices. The vulnerabilities are radially spaced apart on the map 500 include reuse of single use devices after a particular time or procedure with an RF generator at 501, reuse of single use device on multiple RF generators at 502, cloning of single use devices at 503, manufacture of counterfeit devices at 504, reverse engineering, software and firmware, and data at 505, and tampering of data on the electrosurgical puncture device 204 or the RF generator at 506. The amount of protection provided appears in concentric circles of the map 500, such as relatively no protection at a center point 510, low protection 511, medium protection 512, and strong protection 513.

The map 500 illustrates that the asymmetric signature mechanism 320 provides strong protection 513 against manufacture of counterfeit devices at 504, reverse engineering, software and firmware, and data at 505, and tampering of data on the electrosurgical puncture device 204 or the RF generator at 506.

The usage stamp mechanism 322 provides strong protection 513 against reuse of single use devices 501 and reuse of single use device on multiple RF generators 502. The usage stamp mechanism also provides low protection 511 against cloning of single use devices 503.

The challenge-response mechanism 324 provides strong protection cloning of single use devices 503, manufacture of counterfeit devices 504. The usage challenge-response mechanism 324 also provides medium protection 512 against reverse engineering 505 and low protection 511 against device tampering.

As illustrated in the map 500, the particular combination of enforcement mechanisms 308 including the asymmetric signature mechanism 320, the usage stamp mechanism 322, and the challenge-response mechanism 324 combine to provide a sufficiently strong multi-factor authentication scheme for the electrosurgical puncture device 204 against the determined vulnerabilities to single use electrosurgical puncture devices in an efficient manner. The particular combination of enforcement mechanisms is stronger than any single enforcement mechanism or other combinations of enforcement mechanisms, which either are excessive and thus not cost efficient or are inadequate, such as the use of cyclic redundancy checks (CRC), which provide weaker protections for reverse engineering than enforcement mechanisms 308. Further, protections against vulnerabilities are loaded onto the electrosurgical devices, which reduces capital equipment development costs and vulnerabilities if updates for new electrosurgical devices are not maintained.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An electrosurgical puncture device, the electrosurgical puncture device configured for use with an electrosurgical generator in an electrosurgical puncture procedure, the electrosurgical puncture device comprising:
a connector configured to mechanically and electrically couple the electrosurgical puncture device to the electrosurgical generator, the connector including a radiofrequency (RF) conductor to receive an RF puncture signal provided by the electrosurgical generator;
a tangible storage medium electrically coupled to the connector, the storage medium comprising:
data configured for use with the electrosurgical generator in the electrosurgical puncture procedure;
an asymmetric signature mechanism including a digital signature generated from a hash value of the data signed with a private key, the electrosurgical generator including a public key of the asymmetric signature mechanism to verify the hash value;
a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; and
a challenge-response mechanism including a secret in an unreadable region of the storage medium and a challenge engine, the challenge engine configured to calculate a response with the secret and a challenge received from the electrosurgical generator.

2. The electrosurgical puncture device of claim 1, comprising a transseptal puncture device.

3. The electrosurgical puncture device of claim 2, wherein the transseptal puncture device includes one of a transseptal puncture guidewire and a cable couplable to the transseptal puncture guidewire.

4. The electrosurgical puncture device of claim 1, wherein the tangible storage medium includes a secure erasable electronic programmable read only memory (EEPROM).

5. The electrosurgical puncture device of claim 1, wherein the data includes default settings for the electrosurgical generator.

6. The electrosurgical puncture device of claim 1, wherein the digital signature and secret are generated at manufacture of the electrosurgical puncture device.

7. The electrosurgical puncture device of claim 1, wherein the tangible storage medium includes a plurality of at least three enforcement mechanisms including the asymmetric signature mechanism, the usage stamp mechanism, and the challenge-response mechanism.

8. The electrosurgical device of claim 1, wherein the tangible storage medium includes three enforcement mechanisms.

9. The electrosurgical puncture device of claim 1, further comprising a usage count mechanism having a usage amount value in the write protectable region of the storage medium.

10. The electrosurgical puncture device of claim 9, wherein the usage amount value is configured to be one of incremented and decremented with each electrosurgical puncture procedure.

11. The electrosurgical puncture device of claim 1, wherein the electrosurgical generator is configured to apply the asymmetric signature mechanism, the usage stamp mechanism, and the challenge-response mechanism, and permit the electrosurgical puncture procedure to proceed if the electrosurgical puncture device is valid.

12. The electrosurgical puncture device of claim 11, wherein the electrosurgical generator applies two or more of the asymmetric signature mechanism, the usage stamp mechanism, and the challenge-response mechanism concurrently.

13. The electrosurgical puncture device of claim 11, wherein the electrosurgical generator is configured to apply additional enforcement mechanisms.

14. The electrosurgical puncture device of claim 1, wherein the electrosurgical puncture device is included in an electrosurgical puncture assembly including a delivery component associated with the electrosurgical puncture device.

15. An electrosurgical system configured for use in an electrosurgical puncture procedure, the electrosurgical system comprising:
an electrosurgical generator configured to provide a radiofrequency (RF) puncture signal; and
an electrosurgical puncture device, the electrosurgical puncture device configured for use with the electrosurgical generator in the electrosurgical puncture procedure, the electrosurgical puncture device comprising:
a connector configured to mechanically and electrically couple the electrosurgical puncture device to the electrosurgical generator, the connector including an RF conductor to receive the RF puncture signal;
a tangible storage medium electrically coupled to the connector, the storage medium comprising:
data configured for use with the electrosurgical generator in the electrosurgical puncture procedure;
an asymmetric signature mechanism including a digital signature generated from a hash value of the data signed with a private key, the electrosurgical generator including a public key of the asymmetric signature mechanism to verify the hash value;
a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device; and
a challenge-response mechanism including a secret in an unreadable region of the storage medium and a challenge engine, the challenge engine configured to calculate a response with the secret and a challenge received from the electrosurgical generator.

16. The electrosurgical system of claim 15, wherein the electrosurgical puncture device is a transseptal puncture device including one of a transseptal puncture guidewire and a cable couplable to the transseptal puncture guidewire.

17. The electrosurgical puncture system of claim 16, wherein the transseptal puncture device is included in a transseptal puncture assembly including a delivery component associated with the transseptal puncture device.

18. An electrosurgical puncture device, the electrosurgical puncture device configured for use with an electrosurgical generator in an electrosurgical puncture procedure, the electrosurgical puncture device comprising:
a connector configured to mechanically and electrically couple the electrosurgical puncture device to the electrosurgical generator, the connector including a radiofrequency (RF) conductor to receive an RF puncture signal provided by the electrosurgical generator;
a tangible storage medium electrically coupled to the connector, the storage medium comprising:
data configured for use with the electrosurgical generator in the electrosurgical puncture procedure;
an asymmetric signature mechanism including a digital signature generated from a hash value of the data signed with a private key, the electrosurgical generator including a public key of the asymmetric signature mechanism to verify the hash value;
a usage stamp mechanism including a usage stamp having an associated generator code field and a timestamp field in a write protectable region of the storage medium, the usage stamp mechanism configured to provide an input to the electrosurgical generator that ties the electrosurgical puncture device to the electrosurgical generator with a time expiration of the electrosurgical puncture device;
a challenge-response mechanism including a secret in an unreadable region of the storage medium and a challenge engine, the challenge engine configured to calculate a response with the secret and a challenge received from the electrosurgical generator; and
a usage count mechanism having a usage amount value in the write protectable region of the storage medium.

19. The electrosurgical puncture device of claim 18, wherein the electrosurgical generator is configured to apply the asymmetric signature mechanism, the usage stamp mechanism, the challenge-response mechanism, and the usage count mechanism and permit the electrosurgical puncture procedure to proceed if the electrosurgical puncture device is valid.

20. The electrosurgical puncture device of claim 18, wherein the electrosurgical puncture device is included in an electrosurgical puncture system including the electrosurgical generator.
